SEQUENCE LISTING (4 sequences)

SEQ ID NO: 1 - protein, 270 aa, synthetic construct
VTLCNGTYEGLLRRNKVGRNNEKLPTLSGGGGSGGGGYVITTQHWLSGGGGSGGGGFSHQGPAFVTWHRYHLLSGGGGSGGGGSVYDFFVWLHYYSVGGGGSGGGGSLAPIGHNRMYNMVPFFPPVGGGGSGGGGSPAILRRNIHSLSGGGGSGGGGLAFLQYRRLRKGYAPLMETGLSGGGGSGGGGSTFSFRNALEGFDKADGTLDSQVMNLHNLAHSFLNGTNALPHSAANDPVFVGGGGSGGGGSLGYNYAVDLSEEEAPVWSTTL

SEQ ID NO: 2 - protein, 70 aa, synthetic construct
DLVLKRCLLHLAVIGALLAVGATKVPRNQDWLGVSRQLRTKAWNRQLYPEWTEAQRLDCWRGGQVSLKVS

SEQ ID NO: 3 - protein, 257 aa, synthetic construct
FQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLGGGSSGGGSGHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 4 - protein, 211 aa, synthetic construct
TQHWLGLLGPNGTQPQIANCSVYDFFVWLHYYSVRDTLLGPGRPYKAIDFSHQGPAFVTWHRYHLLWLERSGGGGSGGGGSTFSFRNALEGFDKADGTLDSQVMNLHNLAHSFLNGTNALPHSAANDPVFVSGGGGSGGGGDLVLKRCLLHLAVIGALLAVGATKVPRNQDWLGVSRQLRTKAWNRQLYPEWTEAQRLDCWRGGQVSLKVS

(19) United States
(12) Patent Application Publication
(10) Pub. No.: US 2026/0159586 A1
(43) Pub. Date: Jun. 11, 2026

(54) METHODS OF TREATING CANCER WITH A COMBINATION OF A CANCER VACCINE AND A TAAXCD28 BISPECIFIC ANTIGEN-BINDING MOLECULE

(71) Applicant: Regeneron Pharmaceuticals, Inc., Tarrytown, NY (US)

(72) Inventors: David DiLillo, New York, NY (US); Elena Burova, Mount Kisco, NY (US)

(21) Appl. No.: 19/399,807

(22) Filed: Nov. 25, 2025

Related U.S. Application Data
(60) Provisional application No. 63/725,199, filed on Nov. 26, 2024.

(51) Int. Cl. C07K 16/28; A61K 39/00; A61K 45/06; A61P 35/00; C07K 16/30

(52) U.S. Cl. CPC C07K 16/2818; A61K 39/001141; A61K 45/06; A61P 35/00; C07K 16/2896; C07K 16/3069; A61K 2039/505; A61K 2039/53; A61K 2039/54; C07K 2317/31

(57) ABSTRACT

The present disclosure relates to methods of treating or inhibiting the growth of a tumor, wherein the methods include selecting a subject with cancer and administering to the subject in need thereof a therapeutically effective amount of a cancer vaccine (e.g., mRNA vaccine against a tumor) in combination with a bispecific antigen-binding molecule comprising a first antigen-binding domain that binds specifically CD28 and a second antigen-binding domain that binds specifically to a tumor-associated antigen (TAA). The combination therapy demonstrates increased anti-tumor efficacy, increased duration of tumor control and/or increased overall survival, as compared to a subject administered the cancer vaccine as monotherapy.

### SEQUENCE LISTING

The sequence listing of the present application is submitted electronically in xml format with a file name of “SeqList11955.xml,” a creation date of Nov. 11, 2025, and a size of 5,328 bytes. The submitted sequence listing is part of the specification and is hereby incorporated by reference in its entirety.

### FIELD

The present disclosure relates to methods of treating cancer and methods of treating or inhibiting the growth of a tumor by administering a combination of a cancer vaccine and a TAAxCD28 bispecific antigen-binding molecule.

### BACKGROUND

The ability of T-cells to recognize and kill their cellular targets—such as virally-infected cells or tumor cells—depends on a coordinated set of interactions. Foremost among these is the recognition and binding of the target cell by the T-cell Receptor (TCR) complex (which includes the associated CD3 gamma (γ), delta (β), epsilon (ε), and zeta (ζ) chains), and this interaction has been referred to as “signal 1” for T-cell activation. The TCR recognizes a viral or tumor peptide presented on the groove of an MHC protein expressed on the surface of the target cell. Because such binding is generally of low-affinity, successful triggering of “signal 1” requires clustering of many TCR complexes along the interface between the T-cell and its target cell; this interface has been referred to as the “immune synapse”. T-cell activation can be further promoted by additional interactions. For example, T-cells have a molecule referred to as CD28 on their surface, which can provide a co-stimulatory “signal 2” to augment the activation via the TCR complex. When a T-cell recognizes its target cell via its TCR complex, and then also engages “signal 2” via CD28 binding to its cognate ligand(s) on the target cell, T-cell activation is enhanced; as with “signal 1”, CD28-mediated “signal 2” is thought to occur via co-clustering at the immune synapse.

Agonistic anti-CD28 monoclonal antibodies (mAbs) can be applied in sustained ex vivo expansion of cultured T-cells; however, the use of antibodies against CD28 in vivo has been discouraged as a result of a series of acute and serious adverse events in a phase I clinical trial where super agonist anti-CD28 mAb was tested systemically (Hünig, Nat. Rev. Immunol. 2012; 12:317-318). Localized or targeted use of anti-CD28 mAb can be used for promotion of antitumor immunity with less risk (Jung et al., Int J Cancer. 2001 Jan. 15; 91(2):225-30). An antibody that recognizes a tumor-associated antigen (TAA), which is a protein expressed on the surface of tumor cell, can be used to target the anti-CD28 antibody to the tumor microenvironment.

Cancer vaccines are a type of immunotherapeutic that stimulate the adaptive immune response to recognize antigens that are unique to or overexpressed on tumor cells, thereby mobilizing the patient's immune system to attack the tumor. There are a number of categories of cancer vaccines, including cell-based vaccines, such as tumor cells and antigen-presenting cells, and non-cellular vaccines, such as peptide, viral, DNA, and RNA. Development of effective cancer vaccines, however, has been slow and unsteady. The advent of mRNA vaccines, widely known for their use in preventing infection by SARS-CoV-2 by targeting the spike protein of the virus, has spurred generation of several new cancer vaccines, which are only now in the process of being evaluated in clinical trials. As such, their efficacy has not yet been determined.

There remains a need for more effective therapies in treating cancer that achieve substantially improved response without significant side effects.

### SUMMARY

In one aspect, the disclosed technology relates to a method for treating or inhibiting the growth of a tumor, including selecting a subject with cancer; and administering to the subject a therapeutically effective amount of a cancer vaccine in combination with a therapeutically effective amount of a bispecific antigen-binding molecule, wherein the bispecific antigen-binding molecule includes a first antigen-binding domain that binds specifically to a tumor-associated antigen (TAA) and a second antigen-binding domain that binds specifically to CD28, wherein administration of the combination leads to increased anti-tumor efficacy, as compared to a subject administered the cancer vaccine as monotherapy.

In another aspect, the disclosed technology relates to a method for increasing the efficacy of a cancer vaccine, including selecting a subject with cancer; and administering to the subject a therapeutically effective amount of a cancer vaccine in combination with a therapeutically effective amount of a bispecific antigen-binding molecule, wherein the bispecific antigen-binding molecule includes a first antigen-binding domain that binds specifically to a tumor-associated antigen (TAA) and a second antigen-binding domain that binds specifically to CD28, wherein administration of the combination leads to increased anti-tumor efficacy, as compared to a subject administered the cancer vaccine as monotherapy.

In some embodiments, the cancer vaccine comprises mRNA. In some embodiments, the TAA is selected from AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD38, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins, GD2, GD3, GloboH, glypican-3, GM3, gp100, Her2, HLA/B-raf, HLA/k-ras, HLA/MAGE-A3, hTERT, LMP2, MAGE proteins, MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSCA, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

In some embodiments, the cancer is selected from anal cancer, angiosarcoma, basal cell carcinoma, bladder cancer, bone cancer, brain cancer, breast cancer, a B-cell cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, leukemia, liver cancer, lung cancer, lymphoma, melanoma, Merkel cell carcinoma, myeloma, non-small cell lung cancer, ovarian cancer, pancreatic cancer, prostate cancer, rectal cancer, salivary gland cancer, skin cancer, soft tissue sarcoma, stomach cancer, testicular cancer, and uterine cancer. In some embodiments, the cancer vaccine is autogene cevumeran. In some embodiments, the cancer is pancreatic cancer. In some embodiments, the cancer vaccine is nadoferagene firadenovec. In some embodiments, the cancer is bladder cancer. In some embodiments, the cancer vaccine is a personalized cancer mRNA vaccine. In some embodiments, the cancer mRNA vaccine incorporates one or more, subject-specific neoantigens. In some embodiments, the cancer vaccine is selected from BNT113, BNT111, BNT116, BNT122, BNT142, BNT152, BNT153, WGC-043, mRNA-5671N941, mRNA-4157, mRNA-252, and CV9202.

In some embodiments, administration of the combination produces a therapeutic effect selected from one or more of delay in tumor growth, reduction in tumor cell number, increase in tumor regression, prevention of tumor recurrence, prevention or inhibition of metastasis, and inhibition of metastatic tumor growth. In some embodiments, administration of the combination produces a therapeutic effect selected from one or more of increase in duration of survival, increase in likelihood of partial response, increase in likelihood of complete response, and elimination of the need for surgery. In some embodiments, the cancer vaccine is administered to the subject before or concurrently with the administration of the bispecific antigen-binding molecule. In some embodiments, the cancer vaccine is administered to the subject after the administration of the bispecific antigen-binding molecule. In some embodiments, the cancer vaccine is administered intravascularly, subcutaneously, intraperitoneally, or intratumorally. In some embodiments, the bispecific antigen-binding molecule is a bispecific antibody.

In some embodiments, the methods further include administering an additional therapeutic agent or therapy to the subject. In some embodiments, the additional therapeutic agent or therapy is selected from radiation, surgery, a 3-dioxygenase (IDO) inhibitor, an angiopoietin-2 (Ang2) inhibitor, an anti-inflammatory drug, an antibody to a tumor-specific antigen, an antibody-drug conjugate, a B and T lymphocyte attenuator (BTLA) inhibitor, a B7-H3 inhibitor, a B7-H4 inhibitor, Bacillus Calmette-Guerin vaccine, a CD3 inhibitor, a CD47 inhibitor, a chemotherapeutic agent, a cytotoxin, an epidermal growth factor receptor (EGFR) inhibitor, a galectin 9 (GAL9) inhibitor, granulocyte-macrophage colony-stimulating factor (GM-CSF), an IL-10 inhibitor, IL-2, IL-7, IL-12, IL-15, IL-21, indoleamine-2, an interleukin 4 receptor (IL-4R) inhibitor, an interleukin 6 receptor (IL-6R) inhibitor, a Killer-Cell Immunoglobulin-Like Receptor (KIR) inhibitor, a lymphocyte activation gene 3 (LAG3) inhibitor, a T cell immunoglobulin and mucin-domain containing-3 (TIM3) inhibitor, a PD-1 inhibitor, a T cell immunoreceptor with Ig and ITIM domains (TIGIT) inhibitor, a transforming growth factor beta (TGFβ) inhibitor, a V-domain immunoglobulin (Ig)-containing suppressor of T cell activation (VISTA) inhibitor, a vascular endothelial growth factor (VEGF) antagonist, and combinations thereof.

In another aspect, the disclosed technology relates to a method for treating or inhibiting the growth of a tumor, including selecting a subject with cancer; and administering to the subject a therapeutically effective amount of a cancer vaccine in combination with a therapeutically effective amount of a bispecific antigen-binding molecule, wherein the bispecific antigen-binding molecule includes a first antigen-binding domain that binds specifically to a tumor-associated antigen (TAA) and a second antigen-binding domain that binds specifically to CD28, wherein administration of the combination leads to increased overall survival (OS) or increased progression-free survival (PFS), each as compared to an untreated subject or a subject treated with either the cancer vaccine or TAAxCD28 bispecific antibody as monotherapy.

In another aspect, the disclosed technology relates to a kit including a cancer vaccine and a bispecific antigen-binding molecule that includes a first antigen-binding domain that binds specifically to a tumor-associated antigen (TAA) and a second antigen-binding domain that binds specifically to CD28 in combination with written instructions for use of a therapeutically effective amount of the cancer vaccine and the bispecific antigen-binding molecule that includes a first antigen-binding domain that binds specifically to a tumor-associated antigen (TAA) and a second antigen-binding domain that binds specifically to CD28 for inhibiting the growth of a tumor in a subject.

### BRIEF DESCRIPTION OF THE FIGURES

FIGS. 1A-1B relate to Example 1. FIG. 1A is a graph showing amount of IFN gamma (IFNg) released by antigen-specific T cells at Day 0, as number of spot-forming units per million cells (SFU/1×10⁶ splenocytes), as measured by ILISpot assay. FIG. 1B is a graph showing average tumor volume over time in mice administered the indicated vaccines; then implanted with B16F10.9/huBCMA/huCD38 tumor cells; and then administered the indicated bispecific antibodies (bsAb). Arrows indicate days of antibody administration. Proportion of tumor-free mice is indicated for cancer vaccine monotherapy (melanocyte vaccine and control TAAxCD28 bispecific antibody) and for combination therapy (melanocyte vaccine and tumor-specific TAAxCD28 bispecific antibody). Error bars indicate mean+/−SEM.

FIG. 2 relates to Example 2 and is a graph showing average tumor volume over time in mice administered the indicated vaccines; then implanted with B16F10.9/huPSMA tumor cells; and then administered the indicated bsAb. Arrows indicate days of antibody administration. Error bars indicate mean+/−SEM.

FIGS. 3A-3B relate to Example 2 and are graphs showing tumor volume over time of individual mice implanted with B16F10.9/huPSMA tumor cells and then administered vaccines and bsAbs. Arrows indicate days of antibody administration. FIG. 3A shows tumor volume of individual mice administered HPV Vaccine in combination with either Control bsAb or PSMAxCD28 bsAb. FIG. 3B shows tumor volume of individual mice administered Melanocyte Vaccine in combination with either Control bsAb or PSMAxCD28 bsAb.

### DETAILED DESCRIPTION

It is to be understood that the present disclosure is not limited to the particular methods and experimental conditions described, as such methods and conditions may vary. It is also to be understood that the terminology used herein is for the purpose of describing particular embodiments only, and is not intended to be limiting, and that the scope of the present disclosure will be limited only by the appended claims. Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are hereby incorporated by reference in their entirety unless otherwise stated.

The disclosed technology is based, at least in part, on an unexpected discovery that a combination therapy of an anti-cancer vaccine and a tumor associated antigen (TAA) x CD28 co-stimulating bispecific antibody (bsAb) resulted in remarkable anti-tumor immunity in vivo, with TAAxCD28 bsAb enhancing anti-tumor immunity over anti-cancer vaccine monotherapy. Without being bound to a particular theory, it is believed that the cancer vaccine generates tumor-specific T cells, whose antitumor activities are enhanced in the tumor-microenvironment, to which the TAAxCD28 localizes. The presence of the TAAxCD28 bsAb during the priming of a T cell response via vaccination enhances the quantity or quality of the T cell response.

### Methods of Treating Cancer and Methods of Treating or Inhibiting the Growth of a Tumor

The present disclosure includes methods of treating cancer and methods of treating or inhibiting the growth of a tumor by administering to a subject in need thereof a combination therapy comprising a therapeutically effective amount of a cancer vaccine and a therapeutically effective amount of a TAAxCD28 bispecific antigen-binding molecule. The present disclosure also includes methods of increasing the efficacy of a cancer vaccine by administering to a subject with cancer a combination therapy comprising a therapeutically effective amount of a cancer vaccine and a therapeutically effective amount of a TAAxCD28 bispecific antibody.

As used herein, the terms “treating”, “treat”, or the like, mean to alleviate or reduce the severity of at least one symptom or indication, to eliminate the causation of symptoms either on a temporary or permanent basis, to delay or inhibit tumor growth, to reduce tumor cell number, to reduce tumor burden, to promote tumor regression, to cause tumor shrinkage, necrosis and/or disappearance, to prevent tumor recurrence, to prevent or inhibit metastasis, to inhibit metastatic tumor growth, to eliminate the need for surgery, and/or to increase duration of survival of the subject. In many embodiments, the terms “tumor”, “lesion,” “tumor lesion,” “cancer,” and “malignancy” are used interchangeably and refer to one or more cancerous growths.

As used herein, the expression “a subject in need thereof” means a human or non-human mammal that exhibits one or more symptoms or indications of cancer, and/or who has been diagnosed with cancer, and who needs treatment for the same. In some embodiments, the terms “subject” and “patient” are used interchangeably. The expression includes subjects with primary, established, or recurrent tumors (advanced malignancies). In certain embodiments, the expression “a subject in need thereof” includes a subject with a tumor that is resistant to or refractory to or is inadequately controlled by prior therapy (e.g., treatment with an anti-cancer agent).

In certain embodiments, the methods of the present disclosure are used for treating a subject with a solid tumor. As used herein, the term “solid tumor” refers to an abnormal mass of tissue that usually does not contain cysts or liquid areas. Solid tumors may be benign (not cancer) or malignant (cancer). For the purposes of the present disclosure, the term “solid tumor” means malignant solid tumors. The term includes different types of solid tumors named for the cell types that form them, e.g. sarcomas, carcinomas and blastomas. In certain embodiments, the methods of the present disclosure are used for treating a subject with heme tumors. As used herein, the term “heme tumor” refers to cancers affecting blood cells (e.g., B cells or T cells) and includes leukemias, lymphomas and multiple myeloma. Examples of heme tumor include Hodgkin's lymphoma, non-Hodgkin's lymphoma, follicular lymphoma, diffuse large B-cell lymphoma, acute lymphocytic leukemia, chronic lymphocytic leukemia, acute myeloid leukemia, and multiple myeloma.

In an embodiment of the disclosure, the cancer is anal cancer, angiosarcoma, basal cell carcinoma, bladder cancer, bone cancer, brain cancer, breast cancer, a B cell cancer, cervical cancer, cholangiocarcinoma, chondrosarcoma, colon cancer, colorectal cancer, cutaneous squamous cell carcinoma, endometrial cancer, esophageal cancer, glioblastoma multiforme, head & neck squamous cell cancer, hepatocellular carcinoma, kidney cancer, leukemia, liver cancer, lung cancer, lymphoma, melanoma, Merkel cell carcinoma, myeloma, non-small cell lung cancer, ovarian cancer, pancreatic cancer, prostate cancer, rectal cancer, salivary gland cancer, skin cancer, soft tissue sarcoma, stomach cancer, testicular cancer, uterine cancer. In some embodiments, the methods of the present disclosure are used for treating a subject with a TAA-expressing tumor.

In some embodiments, the disclosed methods lead to increased efficacy and duration of anti-tumor response. Methods according to this aspect of the disclosure comprise selecting a subject with cancer and administering to the subject a therapeutically effective amount of a cancer vaccine in combination with a therapeutically effective amount of a TAAxCD28 bispecific antibody. In some embodiments, the disclosed methods provide for increased tumor inhibition and/or increased duration of the anti-tumor response, e.g., by about 20%, more than 20%, more than 30%, more than 40%, more than 50%, more than 60%, more than 70%, or more than 80% as compared to an untreated subject or a subject treated with either the cancer vaccine or TAAxCD28 bispecific antibody as monotherapy. In some embodiments, the disclosed methods increase duration of response in a subject, e.g., by more than 2%, more than 3%, more than 4%, more than 5%, more than 6%, more than 7%, more than 8%, more than 9%, more than 10%, more than 20%, more than 30%, more than 40% or more than 50% more than an untreated subject or a subject treated with either the cancer vaccine or TAAxCD28 bispecific antibody as monotherapy.

In some embodiments, administration of the disclosed combination therapy leads to one or more of: (i) delay in tumor growth and development; (ii) increased disease-free survival (DFS) from date of treatment until recurrence of tumor or death; and (iii) improved overall response rate (ORR), complete response (CR; complete disappearance of all evidence of tumor cells), partial response (PR; at least 30% or more decrease in tumor cells or tumor size), increased overall survival (OS), or increased progression-free survival (PFS), each as compared to an untreated subject or a subject treated with either the cancer vaccine or TAAxCD28 bispecific antibody as monotherapy. Tumor reduction can be measured by known methods, e.g., X-rays, positron emission tomography (PET), computed tomography (CT), magnetic resonance imaging (MRI), cytology, histology, or molecular genetic analyses.

### Cancer Vaccines

The cancer vaccines of the present disclosure may be antigen-presenting cell (APC)-based or antigen-based (e.g., bacterial cells, tumor cells, carbohydrates, or peptides), and the antigens can be introduced through a viral vector or injection of DNA expression plasmid or naked RNA. The first cancer vaccine approved for clinical use was the APC-based vaccine, Sipuleucel-T (Provenge™), for treatment of prostate cancer. An example of a bacterial cell cancer vaccine is TheraCys™, which contains a live attenuated strain of Mycobacterium bovis, Bacillus Calmette-Guerin (BCG), which is implicated in papillary carcinoma. Irradiated tumor cell vaccines have been used to treat hormone-resistant prostate cancer and metastatic melanoma. Carbohydrate-based vaccines involve introduction of a protein carrier fused to a tumor-associated carbohydrate (e.g., the tumor associated carbohydrate antigen-Keyhole limpet hemocyanin (Ag-KHL)) for treating patients with relapsed prostate cancer. An example of a combination DNA and protein vaccine is pDERMATT (plasmid DNA Encoding Recombinant MART-1 and Tetanus toxin fragment-c) for treatment of melanoma. This vaccine contained a DNA plasmid encoding “melanoma antigen recognized by T-cells” (MART-1) and the immunostimulatory Tetanus toxin fragment-c.

The cancer vaccines of the present disclosure may be mRNA vaccines. Humoral and cellular immunity is induced by mRNA vaccines by encoding TAAs, tumor-specific antigens (TSAs), or immunomodulators. Cancer mRNA vaccines that target tumors associated with viruses include vaccines against proteins expressed by Human Papillomavirus (HPV) (e.g., the messenger RNA-HPV therapeutic vaccine (mHTV)) for cervical cancer; Hepatitis B (HBV) for liver cancer; Epstein-Barr (EBV) linked to nasopharyngeal carcinoma, Hodgkin's lymphoma and gastric cancer); human herpesvirus 8, linked Kaposi sarcoma; and HIV (eOD-GT8 60mer), which is associated with a range of different cancers. The tumor-specific antigens may be neo-antigens.

Types of cancer mRNA vaccines currently in clinical trials include RNA-lipid Particle (RNA-LP) Vaccines for Recurrent Pulmonary Osteosarcoma (OSA); Tumor RNA Transfected Dendritic Cell Vaccines for prostate cancer; Messenger RNA (mRNA)-Based, Personalized Cancer Vaccine Against Neoantigens Expressed by the Autologous Cancer for cancers such as Melanoma, Colon Cancer, Gastrointestinal Cancer, Genitourinary Cancer, and Hepatocellular Cancer; and a Prostate Specific Antigen RNA-pulsed dendritic cell vaccine for prostate cancer.

In some embodiments the mRNA vaccines are selected from BNT113, BNT111, BNT116, BNT122, BNT142, BNT152, BNT153, WGC-043, mRNA-5671/V941, mRNA-4157, mRNA-252, and CV9202. In some embodiments the mRNA vaccine is BNT113 for treating HPV16 Driven Carcinomas such as Head and Neck Neoplasms, Cervical Neoplasms, Penile Neoplasms, and Malignant Unknown Primary Tumors. In some embodiments the mRNA vaccine is autogene cevumeran for treating pancreatic cancer. In some embodiments the mRNA vaccine is nadoferagene firadenovec for treating bladder cancer. In some embodiments the mRNA vaccine is basiliximab (Simulect®) for newly diagnosed glioblastoma multiforme.

The cancer mRNA vaccines of the current disclosure may be personalized cancer vaccines. Personalized cancer vaccines increase both the number and antitumor activity of a subject's T cells, such that the subject can mount an effective T cell response that recognizes tumor-specific mutations and/or neoantigens. The tumor mutations and their antigen presenting molecules (i.e., HLA) are unique to each subject, and a personalized antigen/HLA strategy maximizes the personalized immune response. The design of the vaccine, which incorporates one or more subject-specific neoantigens, improves clinical benefit for subjects with a variety of cancer types. In some aspects, the personalized cancer vaccines help to prevent the patient's cancer from recurring by instructing their immune system to better identify cancerous tissue derived from the original cancer lesion.

### TAAXCD28 Bispecific Antigen-Binding Molecules

According to certain exemplary embodiments, the present disclosure includes bispecific antigen-binding molecules that specifically bind a TAA and CD28. Such molecules may be referred to herein as “anti-TAA x anti-CD28” bispecific molecules or other similar terminology (e.g., “TAAxCD28”). The present disclosure provides bispecific antigen-binding molecules constructed with a first antigen-binding arm that binds a TAA and a second antigen-binding arm that binds CD28. In some embodiments, the anti-CD28 arm comprises a heavy chain derived from IGHV3-9*01, IGHJ6*02, IGHD5-12*01.

As used herein, “TAA” refers to a tumor-associated antigen. Non-limiting examples of suitable TAAs include AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD38, CD40, CDK4, CEA, CTLA4, cyclin-B1, CYP1B1, EGFR, EGFRvIII, ErbB2/Her2, ErbB3, ErbB4, ETV6-AML, EpCAM, EphA2, Fra-1, FOLR1, GAGE proteins (i.e., GAGE-1, -2), GD2, GD3, GloboH, glypican-3, GM3, gp100, Her2, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSCA, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

As used herein, the expression “bispecific antigen-binding molecule” refers to a protein, polypeptide or molecular complex comprising at least a first antigen-binding domain and a second antigen-binding domain. Each antigen-binding domain within the bispecific antigen-binding molecule comprises at least one CDR that alone, or in combination with one or more additional CDRs and/or framework regions (FRs), specifically binds to a particular antigen. In the context of the present disclosure, the first antigen-binding domain specifically binds a first antigen (e.g., a TAA such as CD38), and the second antigen-binding domain specifically binds a second, distinct antigen (e.g., CD28).

The first antigen-binding domain and the second antigen-binding domain may be directly or indirectly connected to one another to form a bispecific antigen-binding molecule of the present disclosure. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

In some embodiments, the bispecific antigen-binding molecule is a bispecific antibody or antigen-binding fragment thereof. Each antigen-binding domain of a bispecific antibody or antigen-binding fragment thereof comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

As used herein, “isolated” antigen-binding molecules (e.g., antibodies or antigen-binding fragments thereof) refer to polypeptides, polynucleotides and vectors, which are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antigen-binding proteins (e.g., antibodies or antigen-binding fragments).

As used herein, an “antibody” refers to an immunoglobulin molecule comprising four polypeptide chains, two heavy chains (HC) and two light chains (LC) inter-connected by disulfide bonds. Each heavy chain (HC) comprises a heavy chain variable region (abbreviated herein as HCVR or V_{H}) and a heavy chain constant region (e.g., IgG, IgG1 or IgG4). The heavy chain constant region comprises three domains, C_{H}1, C_{H}2 and C_{H}3. Each light chain (LC) comprises a light chain variable region (abbreviated herein as LCVR or V_{L}) and a light chain constant region (e.g., lambda or kappa). The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} includes three CDRs and four FRs, arranged from amino-terminus to carboxy-terminus in the following order: FR1, CDR1, FR2, CDR2, FR3, CDR3, FR4. A heavy chain CDR may be referred to as HCDR and a light chain CDR may be referred to as LCDR. In different embodiments, the FRs of an antibody (or antigen-binding portion thereof) may be identical to the human germline sequences, or may be naturally or artificially modified.

As used herein, an “antigen-binding arm” of a Y-shaped IgG antibody (e.g., a CD28 binding arm) refers to a structural portion of the antibody that confers binding specificity to the antigen. For example, an antigen-binding arm of an IgG antibody has a heavy chain (HC) associated with a light chain (LC).

As used herein, an “antigen-binding portion” of an antibody, “antigen-binding fragment” of an antibody, and the like include any naturally occurring, enzymatically obtainable, synthetic, or genetically engineered polypeptide or glycoprotein that specifically binds an antigen to form a complex. A multispecific antigen-binding fragment of an antibody binds to multiple antigens (e.g., two different antigens if the fragment is bispecific). Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments.

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}—C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2, (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As used herein, the term “specifically binds,” or “binds specifically” or the like, means that an antigen-binding molecule (e.g. an antibody or antigen-binding fragment thereof) forms a complex with an antigen that is relatively stable under physiologic conditions. Methods for determining whether an antibody specifically binds to an antigen are well known in the art and include, for example, equilibrium dialysis, surface plasmon resonance, and the like. Antigen-binding proteins (e.g., antibodies or antigen-binding fragments thereof) that specifically bind an antigen have a binding affinity to the antigen, such as a TAA or CD28 protein, expressed as K_{D}, of less than about 10⁻⁶ M (e.g., 10⁻⁷ M, 10⁻⁸ M, 10⁻⁹ M, 10⁻¹⁰ M, 10⁻¹¹ M, or 10⁻¹² M), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. “Anti-TAA” refers to an antigen-binding protein (or other molecule such as an antigen-binding arm), for example an antibody or antigen-binding fragment thereof, that binds specifically to a TAA and “anti-CD28” refers to an antigen-binding protein (or other molecule such as an antigen-binding arm), for example an antibody or antigen-binding fragment thereof, that binds specifically to CD28. “TAAxCD28” refers to refers to an antigen-binding protein (or other molecule), for example an antibody or antigen-binding fragment thereof, that binds specifically to a TAA and to CD28 (and, optionally, to one or more other antigens).

According to certain embodiments, the bispecific antibodies may be constructed utilizing a heavy chain from an anti-CD28 antibody, a heavy chain from an anti-TAA antibody and a common light chain from the anti-TAA antibody. In other instances, the bispecific antibodies may be constructed utilizing a heavy chain from an anti-CD28 antibody, a heavy chain from an anti-TAA antibody and a light chain from an anti-CD28 antibody or an antibody light chain known to be promiscuous or pair effectively with a variety of heavy chain arms. Bispecific antibodies may be manufactured having an IgG1 Fc domain or a modified (chimeric) IgG4 Fc domain as set forth in U.S. Pat. No. 9,359,437.

As used herein, “CD28” refers to the human CD28 protein unless specified as being from a non-human species (e.g., “mouse CD28,” “monkey CD28,” etc.). CD28 is expressed on T cells as a costimulatory receptor. In an embodiment of the disclosure, human CD28 comprises the amino acid sequence as set forth in NCBI accession No. NP_006130.1. In one embodiment, human CD28 is expressed with a C-terminal murine Fc tag (hCD28.mFc). As used herein, “an antibody that binds CD28” or an “anti-CD28 antibody” includes antibodies and antigen-binding fragments thereof that specifically recognize CD28.

All references to proteins, polypeptides and protein fragments herein are intended to refer to the human version of the respective protein, polypeptide or protein fragment unless explicitly specified as being from a non-human species. Thus, the expression “CD38” or “CD28” means human CD38 or human CD28, respectively, unless specified as being from a non-human species, e.g., “mouse CD38,” “mouse CD28,” “monkey CD38,” “monkey CD2,” etc.

Bispecific antigen-binding molecules (e.g., bispecific antibodies) may have an effector arm that binds to a first antigen and a targeting arm that binds to second antigen. The effector arm may be the first antigen-binding domain (e.g., anti-CD28) that binds to the antigens on effector cells (e.g., T cells). The targeting arm may be the second antigen-binding domain (e.g., anti-TAA antibody) that binds to the antigens on target cells (e.g., tumor cells or immune cells). In the context of the present disclosure, the effector arm binds to CD28 and the targeting arm binds to a TAA.

The antibodies of the present disclosure may function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the present disclosure in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337; Clynes et al., 1998, Proc. Natl. Acad. Sci. (USA), 95:652-656.) The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

In certain embodiments, the TAAxCD28 bispecific antibodies of the present disclosure are human antibodies. As used herein, the term “human antibody” is intended to include antibodies having variable and constant regions derived from human germline immunoglobulin sequences. The human antibodies of the present disclosure may include amino acid residues not encoded by human germline immunoglobulin sequences (e.g., mutations introduced by random or site-specific mutagenesis in vitro or by somatic mutation in vivo), for example in the CDRs and in particular CDR28. However, the term “human antibody”, as used herein, is not intended to include antibodies in which CDR sequences derived from the germline of another mammalian species, such as a mouse, have been grafted onto human framework sequences.

The antibodies used in the methods disclosed herein may be recombinant human antibodies. As used herein, the term “recombinant human antibody” includes all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The antibodies of the present disclosure may be bispecific, or multispecific. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. The TAAxCD28 bispecific antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second or additional binding specificity. As used herein, the expression “anti-CD28 antibody” includes both monospecific anti-CD28 antibodies as well as multispecific (e.g., bispecific) antibodies or antigen-binding molecules comprising a TAA-binding arm and a second arm that binds CD28.

The present disclosure encompasses antigen-binding molecules having amino acid sequences that vary from those of the exemplary molecules disclosed herein but that retain the ability to bind CD28 and/or a TAA. Such variant molecules may comprise one or more additions, deletions, or substitutions of amino acids when compared to parent sequence, but exhibit biological activity that is essentially equivalent to that of the described bispecific antigen-binding molecules. The present disclosure includes antigen-binding molecules that are bioequivalent to any of the exemplary antigen-binding molecules set forth herein. As used herein, the term “bioequivalent” refers to TAAxCD28 bispecific antibodies or fragments thereof that are pharmaceutical equivalents or pharmaceutical alternatives whose rate and/or extent of absorption do not show a significant difference with that of a reference antibody when administered at the same molar dose under similar experimental conditions, either single dose or multiple dose. In the context of the present disclosure, the term “bioequivalent” includes antigen-binding proteins that bind to a TAA and CD28 and do not have clinically meaningful differences with the reference antibody with respect to safety, purity and/or potency.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

As used herein, the term “recombinant” antigen-binding proteins, such as antibodies or antigen-binding fragments thereof, refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a host cell (e.g., Chinese hamster ovary (CHO) cell) or cellular expression system or isolated from a recombinant combinatorial human antibody library. The present disclosure includes recombinant antigen-binding proteins as set forth herein.

Antigen-binding domains specific for particular antigens can be prepared by any antibody generating technology known in the art. Once obtained, two different antigen-binding domains, specific for two different antigens (e.g., a TAA and CD28), can be appropriately arranged relative to one another to produce a bispecific antigen-binding molecule of the present disclosure using routine methods. (A discussion of exemplary bispecific antibody formats that can be used to construct the bispecific antigen-binding molecules of the present disclosure is provided elsewhere herein). In certain embodiments, one or more of the individual components (e.g., heavy and light chains) of the multispecific antigen-binding molecules of the disclosure are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., a TAA or CD28) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules of the present disclosure.

Genetically engineered animals may be used to make human bispecific antigen-binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. (See, e.g., US 2011/0195454 for a detailed discussion of such engineered mice and the use thereof to produce bispecific antigen-binding molecules).

### Combination Therapies

In general, the methods of the present disclosure include administering a combination therapy including a therapeutically effective amount of a cancer vaccine (e.g., a melanocyte mRNA vaccine) in combination with a therapeutically effective amount of a TAAxCD28 costimulatory bsAb (e.g., CD38xCD28). As used herein, the expression “in combination with” means that a first disclosed therapy is administered before, after, or concurrently with a second disclosed therapy. This expression includes sequential or concurrent administration of the therapies.

In some embodiments, the disclosed combination therapy treats or inhibits the growth of a tumor in a subject. In some embodiments, administration of the disclosed TAAxCD28 bispecific antibody increases the efficacy of the cancer vaccine. In some embodiments, the combined administration of the cancer vaccine and TAAxCD28 bispecific antibody with an additional therapeutic agent or therapy leads to improved anti-tumor efficacy, reduced side effects of one or both of the primary therapies, and/or reduced dosage of one or both of the primary therapies.

In some embodiments, the disclosed methods further include administration of an additional therapeutic agent or therapy. The additional therapeutic agent or therapy may include one or more of radiation, surgery, a 3-dioxygenase (IDO) inhibitor, an angiopoietin-2 (Ang2) inhibitor, an anti-inflammatory drug, an antibody to a tumor-specific antigen, an antibody-drug conjugate, a B and T lymphocyte attenuator (BTLA) inhibitor, a B7-H3 inhibitor, a B7-H4 inhibitor, Bacillus Calmette-Guerin vaccine, a CD3 inhibitor, a CD47 inhibitor, a chemotherapeutic agent, a cytotoxin, an epidermal growth factor receptor (EGFR) inhibitor, a galectin 9 (GAL9) inhibitor, granulocyte-macrophage colony-stimulating factor (GM-CSF), an IL-10 inhibitor, IL-2, IL-7, IL-12, IL-15, IL-21, indoleamine-2, an interleukin 4 receptor (IL-4R) inhibitor, an interleukin 6 receptor (IL-6R) inhibitor, a Killer-Cell Immunoglobulin-Like Receptor (KIR) inhibitor, a lymphocyte activation gene 3 (LAG3) inhibitor, a PD-1 inhibitor, a T cell immunoglobulin and mucin-domain containing-3 (TIM3) inhibitor, a T cell immunoreceptor with Ig and ITIM domains (TIGIT) inhibitor, a transforming growth factor beta (TGFβ) inhibitor, a V-domain immunoglobulin (Ig)-containing suppressor of T cell activation (VISTA) inhibitor, and a vascular endothelial growth factor (VEGF) antagonist.

The present disclosure also provides kits comprising the disclosed cancer vaccine and TAAxCD28 bispecific antibody for therapeutic uses as described herein. Kits typically include a label indicating the intended use of the contents of the kit and instructions for use. As used herein, the term “label” includes any writing, or recorded material supplied on, in or with the kit, or that otherwise accompanies the kit. In some embodiments, the present disclosure provides a kit for treating a subject afflicted with cancer, wherein the kit includes: (a) a therapeutically effective dose of a disclosed cancer vaccine; a therapeutically effective dose of a disclosed TAAxCD28 bispecific antibody; and (b) instructions for using the combination of doses in any of the methods disclosed herein.

### Administration Regimens

The present disclosure includes methods that comprise administering to a subject with cancer a combination of the cancer vaccine and TAAxCD28 bispecific antibody at a dosing frequency that achieves a therapeutic response. In some embodiments, the cancer vaccine and/or the TAAxCD28 bispecific antibody is administered to the subject in one or more doses administered about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, when the cancer vaccine is administered “before” the TAAxCD28 bispecific antibody, the cancer vaccine may be administered more than 12 weeks, about 12 weeks, about 11 weeks, about 10 weeks, about 9 weeks, about 8 weeks, about 7 weeks, about 6 weeks, about 5 weeks, about 4 weeks, about 3 weeks, about 2 weeks, about 1 week, about 150 hours, about 100 hours, about 72 hours, about 60 hours, about 48 hours, about 36 hours, about 24 hours, about 12 hours, about 10 hours, about 8 hours, about 6 hours, about 4 hours, about 2 hours, about 1 hour, or about 30 minutes prior to the administration of the TAAxCD28 bispecific antibody.

In some embodiments, when the cancer vaccine is administered “after” the TAAxCD28 bispecific antibody, the cancer vaccine may be administered about 30 minutes, about 1 hour, about 2 hours, about 4 hours, about 6 hours, about 8 hours, about 10 hours, about 12 hours, about 24 hours, about 36 hours, about 48 hours, about 60 hours, about 72 hours, about 1 week, about 2 weeks, about 3 weeks, about 4 weeks, about 5 weeks, about 5 weeks, about 7 weeks, about 8 weeks, about 9 weeks, about 10 weeks, about 11 weeks, about 12 weeks, or more than 12 weeks after the administration of the TAAxCD28 bispecific antibody.

As used herein, “concurrent” administration means that the cancer vaccine and TAAxCD28 bispecific antibody are administered to the subject in a single dosage form (e.g., co-formulated, as appropriate) or in separate dosage forms administered to the subject within about 30 minutes or less of each other (i.e., before, after, or at the same time), such as about 15 minutes or less, or about 5 minutes or less. If administered in separate dosage forms, each dosage form may be administered via the same route (e.g., both administered intravenously, subcutaneously, etc.); or, alternatively, each dosage form may be administered via a different route. In any event, administering the therapies in a single dosage from, in separate dosage forms by the same route, or in separate dosage forms by different routes are all considered “concurrent” administration” for purposes of the present disclosure.

As used herein, “sequential” administration means that each dose of a selected therapy is administered to the subject at a different point in time, e.g., on different days separated by a predetermined interval (e.g., hours, days, weeks, or months). For illustrative purposes, sequential administration may include administering an initial dose of the cancer vaccine (or TAAxCD28 bispecific antibody), followed by one or more secondary doses the TAAxCD28 bispecific antibody (or cancer vaccine), optionally followed by one or more tertiary doses of the TAAxCD28 bispecific antibody (or cancer vaccine).

### Pharmaceutical Compositions and Dosage

With respect to pharmaceutical compositions, the disclosed cancer vaccine or TAAxCD28 bispecific antibody may each be formulated with one or more carriers, excipients and/or diluents. Pharmaceutical compositions comprising the cancer vaccine and/or TAAxCD28 bispecific antibody may be formulated for specific uses, such as for veterinary uses or pharmaceutical uses in humans. The form of the composition (e.g., dry powder, liquid formulation, etc.) and the excipients, diluents and/or carriers used will depend upon the intended therapeutic use and desired mode of administration of the cancer vaccine or TAAxCD28 bispecific antibody.

A pharmaceutical composition of the present disclosure may contain either or both of the cancer vaccine and TAAxCD28 bispecific antibody. Such pharmaceutical compositions may be administered to a subject by a variety of routes such as orally, transdermally, subcutaneously, intranasally, intravascularly, intravenously, intramuscularly, intraperitoneally, intratumorally, intrathecally, topically, or locally. In some embodiments, the pharmaceutical composition is administered to the subject intravascularly, subcutaneously, intraperitoneally, or intratumorally. Pharmaceutical compositions can be conveniently presented in unit dosage forms containing a predetermined amount of the disclosed cancer vaccine and/or TAAxCD28 bispecific antibody per dose.

In general, the amount of cancer vaccine and/or TAAxCD28 bispecific antibody administered to a subject according to the methods of the present disclosure is a therapeutically effective amount. As used herein, “therapeutically effective amount” means an amount of the cancer vaccine in combination with the TAAxCD28 bispecific antibody that results in one or more of: (a) a reduction in the severity or duration of a symptom of a cancer; (b) enhanced inhibition of tumor growth, or an increase in tumor necrosis, tumor shrinkage and/or tumor disappearance; (c) delay in tumor growth and development; (d) inhibition or retardation or termination of tumor metastasis; (e) prevention of recurrence of tumor growth; (f) increase in survival of a subject with a cancer; and (g) a reduction in the use or need for conventional anti-cancer therapy (e.g., reduced or eliminated use of chemotherapeutic or cytotoxic agents) as compared to an untreated subject or a subject treated with a cancer vaccine as monotherapy.

In some embodiments, a therapeutically effective amount of the cancer vaccine may be from about 0.1 μg to about 1000 μg (micrograms), or about 1 μg to about 500 μg, or about 0.01 μg/kg to about 200 μg/kg, or from about 0.05 μg/kg to about 100 μg/kg of the subject's body weight. In some embodiments, a therapeutically effective amount of the TAAxCD28 bispecific antibody may be from about 0.05 mg to about 2000 mg, or about 0.5 mg to about 50 mg, or from about 0.1 mg/kg to about 50 mg/kg, or from about 1 mg/kg to about 20 mg/kg of the subject's body weight.

As used herein, the singular forms “a,” “an,” and “the” include plural reference unless the context clearly dictates otherwise. As used herein, the terms “including,” “comprising,” “containing,” or “having” and variations thereof are meant to encompass the items listed thereafter and equivalents thereof as well as additional subject matter unless otherwise noted. As used herein, the phrases “in one embodiment,” “in various embodiments,” “in some embodiments,” and the like are used repeatedly. Such phrases do not necessarily refer to the same embodiment, but they may unless the context dictates otherwise. As used herein, the terms “and/or” or “/” means any one of the items, any combination of the items, or all of the items with which this term is associated.

### EXAMPLES

The following examples are put forth so as to provide those of ordinary skill in the art with a complete disclosure and description of how to make and use the methods and compositions of the present disclosure and are not intended to limit the scope of what the inventors regard as their invention. Likewise, the disclosure is not limited to any particular preferred embodiments described herein. Indeed, modifications and variations of the embodiments may be apparent to those skilled in the art upon reading this specification and can be made without departing from its spirit and scope. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Potent Anti-Tumor Efficacy of a Combination of Melanocyte Antigen mRNA Vaccine and TAAxCD28 Bispecific Antibody

This Example relates to an in vivo study demonstrating that combination therapy of a melanocyte antigen mRNA vaccine and a huCD38xhuCD28 bispecific antibody drives potent anti-tumor response in vivo. Melanocyte antigen mRNA vaccination stimulates weak immunity against B16 tumors. The combination therapy leads to improved control of tumor growth, as compared to the melanocyte antigen vaccine as monotherapy.

To determine the in vivo anti-tumor efficacy of a vaccine in combination with a huCD38xhuCD28 bispecific antibody (bsAb), a syngeneic tumor study was performed using B16F10.9/huBCMA/huCD38 tumor cells, which is a mouse model of melanoma. C57BL/6 mice used for this study were engineered to express human CD28 (huCD28) and huCD38 (in addition to huCD3, hu4-1BB, huBCMA, and huSLAMF7) via Velocigene technology, as previously described (Valenzuela, 2003). The B16F10.9/huBCMA/huCD38 tumor cells utilized in this study endogenously express the murine (mu) melanocyte antigens muTRP-2 and muPMEL, and were engineered to express huCD38 cell-surface protein, which represents a tumor-specific antigen that is recognized by the huCD38xhuCD28 antibody. The melanocyte mRNA vaccine utilized in the study encodes immunogenic epitopes from muTRP-2 and huPMEL that stimulate T cell responses against muTRP-2 and muPMEL, which are presented by B16F10.9/huBCMA/huCD38 tumor cells.

8-10-week old mice (4-5 mice per group) were vaccinated prophylactically with the melanocyte mRNA vaccine 3 times, with each dose separated by 7 days. The vaccine was delivered in two parts that administered on two consecutive days. On the first day, mRNA encoding immunogenic epitopes from muTRP-2 (9 minigenes from muTRP-2) was administered i.v. (retro-orbitally). On the second day, mRNA encoding immunogenic epitopes from huPMEL (1 minigene from huPMEL) was administered i.v. (retro-orbitally). Thus, the muTRP-2 vaccine was administered on Day (−21), Day (−14), and Day (−7), and the huPMEL vaccine was administered on Day (−20), Day (−13) and Day (−6). As a negative control, an mRNA vaccine expressing immunogenic epitopes from the Human Papilloma Virus (HPV) oncogenes E6 and E7 was administered at the same schedule. On Day 0, mice were injected subcutaneously with 3×10⁵ B16F10.9/huBCMA/huCD38 tumor cells. On Day 1, the mice were administered either a huCD28-binding control bsAb or a huCD38xhuCD28 bsAb at 10 mg/kg intraperitoneally (i.p.). The mice received five more doses of these bispecific antibodies on Days 5, 9, 12, 15, and 18, for a total of six doses. Tumor growth was assessed twice weekly over 30 days by caliper measurement, and tumor volume was calculated by the using the following formula: (length×width2)/2. Splenocytes were collected at Day 0 and IFNγ was measured by ELISpot assay.

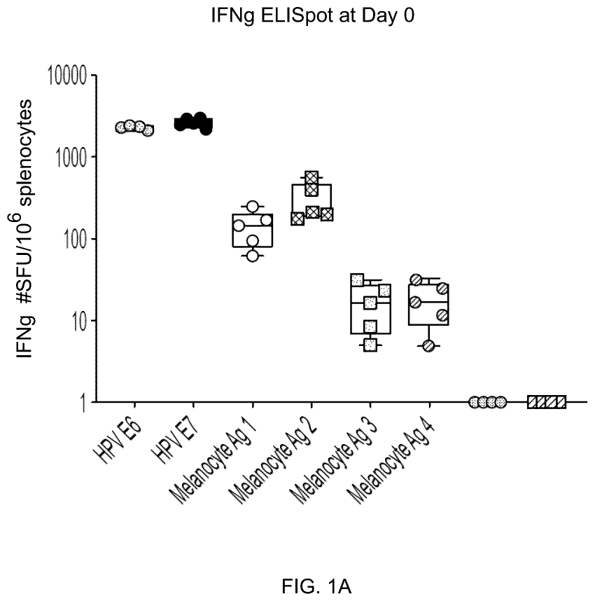

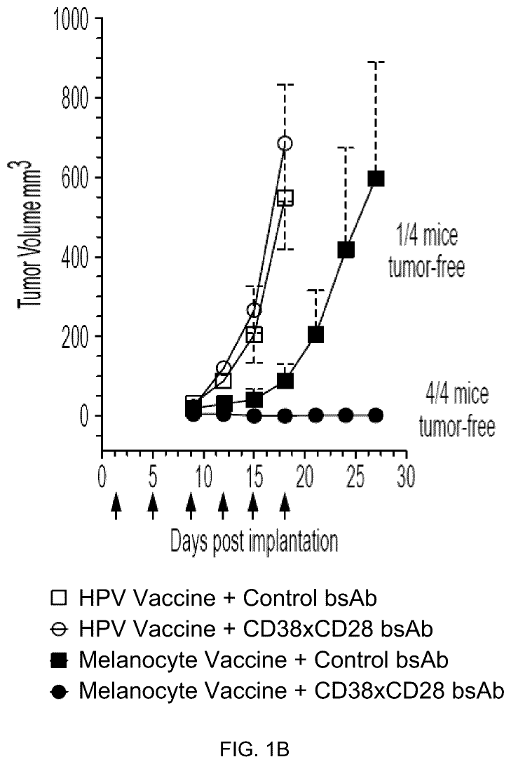

HPV control vaccination against E6 and E7 led to strong T cell responses, while vaccination against melanocyte antigens drove modest T cell responses (FIG. 1A). Both mice administered HPV control vaccine with bsAb control and mice administered HPV control vaccine with huCD38xhuCD28 bsAb showed rapid tumor growth, with average tumor volumes ranging between about 550 mm³ and 700 mm³ by Day 18 (Table 4, FIG. 1B). Mice administered melanocyte antigen vaccine as monotherapy (i.e., melanocyte antigen vaccine with bsAb control) showed some control of tumor growth, with an average tumor volume of about 600 mm³ at Day 27 (Table 7) and 1 out of 4 mice tumor-free at end of experiment at Day 30 (FIG. 1B). Mice administered combination therapy of melanocyte antigen vaccine and huCD38xhuCD28 bsAb showed complete control of tumor growth by Day 15 (Tables 1-7), with 4 out of 4 mice tumor-free at end of experiment at Day 30 (FIG. 1B).

Tables 1-7 set forth tumor growth and survival at Days 9, 12, 15, 18, 21, 24, and 27 respectively.

TABLE 1
Tumor Growth and Survival at Day 9

| | Mean Tumor | Tumor Volume SEM | Number mice |
|---|---|---|---|
| Vaccine and Antibody | Volume (mm³) | (mm³) | still alive |
| HPV control vaccine + | 27.73 | 6.76 | 5 out of 5 |
| CD28-binding bsAb (10 mg/kg) | | | |
| HPV control vaccine + | 17.81 | 5.36 | 5 out of 5 |
| CD38 × CD28 bsAb (10 mg/kg) | | | |
| Melanocyte vaccine + | 14.49 | 6.39 | 4 out of 4 |
| CD28-binding bsAb (10 mg/kg) | | | |
| Melanocyte vaccine + | 2.00 | 0.98 | 4 out of 4 |
| CD38 × CD28 bsAb (10 mg/kg) | | | |

TABLE 2
Tumor Growth and Survival at Day 12

| | Mean Tumor | Tumor Volume SEM | Number mice |
|---|---|---|---|
| Vaccine and Antibody | Volume (mm³) | (mm³) | still alive |
| HPV control vaccine + | 87.97 | 11.38 | 5 out of 5 |
| CD28-binding bsAb (10 mg/kg) | | | |
| HPV control vaccine + | 117.56 | 18.24 | 5 out of 5 |
| CD38 × CD28 bsAb (10 mg/kg) | | | |
| Melanocyte vaccine + | 29.16 | 12.01 | 4 out of 4 |
| CD28-binding bsAb (10 mg/kg) | | | |
| Melanocyte vaccine + | 2.00 | 1.16 | 4 out of 4 |
| CD38 × CD28 bsAb (10 mg/kg) | | | |

TABLE 3
Tumor Growth and Survival at Day 15

| | Mean Tumor | Tumor Volume SEM | Number mice |
|---|---|---|---|
| Vaccine and Antibody | Volume (mm³) | (mm³) | still alive |
| HPV control vaccine + | 205.04 | 72.00 | 5 out of 5 |
| CD28-binding bsAb (10 mg/kg) | | | |
| HPV control vaccine + | 265.73 | 58.92 | 5 out of 5 |
| CD38 × CD28 bsAb (10 mg/kg) | | | |
| Melanocyte vaccine + | 39.28 | 25.75 | 4 out of 4 |
| CD28-binding bsAb (10 mg/kg) | | | |
| Melanocyte vaccine + | 0.00 | 0.00 | 4 out of 4 |
| CD38 × CD28 bsAb (10 mg/kg) | | | |

TABLE 4
Tumor Growth and Survival at Day 18

| | Mean Tumor | Tumor Volume | Number mice |
|---|---|---|---|
| Vaccine and Antibody | Volume (mm³) | SEM (mm³) | still alive |
| HPV control vaccine + | 547.43 | 129.10 | 5 out of 5 |
| CD28-binding bsAb (10 mg/kg) | | | |
| HPV control vaccine + | 684.56 | 149.4 | 5 out of 5 |
| CD38 × CD28 bsAb (10 mg/kg) | | | |
| Melanocyte vaccine + | 86.92 | 44.38 | 4 out of 4 |
| CD28-binding bsAb (10 mg/kg) | | | |
| Melanocyte vaccine + | 0.00 | 0.00 | 4 out of 4 |
| CD38 × CD28 bsAb (10 mg/kg) | | | |

TABLE 5
Tumor Growth and Survival at Day 21

| | Mean Tumor | Tumor Volume SEM | Number mice |
|---|---|---|---|
| Vaccine and Antibody | Volume (mm³) | (mm³) | still alive |
| HPV control vaccine + | N/A | N/A | 4 out of 5 |
| CD28-binding bsAb (10 mg/kg) | | | |
| HPV control vaccine + | N/A | N/A | 4 out of 5 |
| CD38 × CD28 bsAb (10 mg/kg) | | | |
| Melanocyte vaccine + | 205.56 | 109.90 | 4 out of 4 |
| CD28-binding bsAb (10 mg/kg) | | | |
| Melanocyte vaccine + | 0.00 | 0.00 | 4 out of 4 |
| CD38 × CD28 bsAb (10 mg/kg) | | | |

TABLE 6
Tumor Growth and Survival at Day 24

| | Mean Tumor | Tumor Volume SEM | Number mice |
|---|---|---|---|
| Vaccine and Antibody | Volume (mm³) | (mm³) | still alive |
| HPV control vaccine + | N/A | N/A | 3 out of 5 |
| CD28-binding bsAb (10 mg/kg) | | | |
| HPV control vaccine + | N/A | N/A | 4 out of 5 |
| CD38 × CD28 bsAb (10 mg/kg) | | | |
| Melanocyte vaccine + | 417.94 | 255.1 | 4 out of 4 |
| CD28-binding bsAb (10 mg/kg) | | | |
| Melanocyte vaccine + | 0.00 | 0.00 | 4 out of 4 |
| CD38 × CD28 bsAb (10 mg/kg) | | | |

TABLE 7
Tumor Growth and Survival at Day 27

| | Mean Tumor | Tumor Volume SEM | Number mice |
|---|---|---|---|
| Vaccine and Antibody | Volume (mm³) | (mm³) | still alive |
| HPV control vaccine + | N/A | N/A | 1 out of 5 |
| CD28-binding bsAb (10 mg/kg) | | | |
| HPV control vaccine + | N/A | N/A | 2 out of 5 |
| CD38 × CD28 bsAb (10 mg/kg) | | | |
| Melanocyte vaccine + | 597.74 | 292.8 | 4 out of 4 |
| CD28-binding bsAb (10 mg/kg) | | | |
| Melanocyte vaccine + | 0.00 | 0.00 | 4 out of 4 |
| CD38 × CD28 bsAb (10 mg/kg) | | | |

The study was repeated with a greater number of mice. Tables 8-15 set forth the tumor growth and survival in the second study.

TABLE 8
Tumor Growth and Survival at Day 5

| | Mean Tumor | Tumor Volume SEM | Number mice |
|---|---|---|---|
| Vaccine and Antibody | Volume (mm³) | (mm³) | still alive |
| HPV control vaccine + | 21.90 | 2.58 | 10 out of 10 |
| CD28-binding bsAb (10 mg/kg) | | | |
| HPV control vaccine + | 27.63 | 3.53 | 10 out of 10 |
| CD38 × CD28 bsAb (10 mg/kg) | | | |
| Melanocyte vaccine + | 24.81 | 5.66 | 10 out of 10 |
| CD28-binding bsAb (10 mg/kg) | | | |
| Melanocyte vaccine + | 30.14 | 4.58 | 11 out of 11 |
| CD38 × CD28 bsAb (10 mg/kg) | | | |

TABLE 9
Tumor Growth and Survival at Day 9

| | Mean Tumor | Tumor Volume SEM | Number mice |
|---|---|---|---|
| Vaccine and Antibody | Volume (mm³) | (mm³) | still alive |
| HPV control vaccine + | 66.59 | 6.87 | 10 out of 10 |
| CD28-binding bsAb (10 mg/kg) | | | |
| HPV control vaccine + | 53.82 | 4.82 | 10 out of 10 |
| CD38 × CD28 bsAb (10 mg/kg) | | | |
| Melanocyte vaccine + | 52.25 | 5.64 | 10 out of 10 |
| CD28-binding bsAb (10 mg/kg) | | | |
| Melanocyte vaccine + | 53.58 | 5.36 | 11 out of 11 |
| CD38 × CD28 bsAb (10 mg/kg) | | | |

TABLE 10
Tumor Growth and Survival at Day 11

| | Mean Tumor | Tumor Volume SEM | Number mice |
|---|---|---|---|
| Vaccine and Antibody | Volume (mm³) | (mm³) | still alive |
| HPV control vaccine + | 76.66 | 9.68 | 10 out of 10 |
| CD28-binding bsAb (10 mg/kg) | | | |
| HPV control vaccine + | 49.33 | 6.50 | 10 out of 10 |
| CD38 × CD28 bsAb (10 mg/kg) | | | |
| Melanocyte vaccine + | 72.93 | 8.28 | 10 out of 10 |
| CD28-binding bsAb (10 mg/kg) | | | |
| Melanocyte vaccine + | 49.88 | 3.80 | 11 out of 11 |
| CD38 × CD28 bsAb (10 mg/kg) | | | |

TABLE 11
Tumor Growth and Survival at Day 15

| | Mean Tumor | Tumor Volume SEM | Number mice |
|---|---|---|---|
| Vaccine and Antibody | Volume (mm³) | (mm³) | still alive |
| HPV control vaccine + | 55.98 | 19.19 | 10 out of 10 |
| CD28-binding bsAb (10 mg/kg) | | | |
| HPV control vaccine + | 113.56 | 45.83 | 10 out of 10 |
| CD38 × CD28 bsAb (10 mg/kg) | | | |
| Melanocyte vaccine + | 63.78 | 22.67 | 10 out of 10 |
| CD28-binding bsAb (10 mg/kg) | | | |
| Melanocyte vaccine + | 3.23 | 1.92 | 11 out of 11 |
| CD38 × CD28 bsAb (10 mg/kg) | | | |

TABLE 12
Tumor Growth and Survival at Day 18

| | Mean Tumor | Tumor Volume | Number mice |
|---|---|---|---|
| Vaccine and Antibody | Volume (mm³) | SEM (mm³) | still alive |
| HPV control vaccine + | 142.72 | 55.32 | 10 out of 10 |
| CD28-binding bsAb (10 mg/kg) | | | |
| HPV control vaccine + | 225.64 | 74.61 | 10 out of 10 |
| CD38 × CD28 bsAb (10 mg/kg) | | | |
| Melanocyte vaccine + | 184.32 | 89.97 | 10 out of 10 |
| CD28-binding bsAb (10 mg/kg) | | | |
| Melanocyte vaccine + | 0.00 | 0.00 | 11 out of 11 |
| CD38 × CD28 bsAb (10 mg/kg) | | | |

TABLE 13
Tumor Growth and Survival at Day 22

| | Mean Tumor | Tumor Volume SEM | Number mice |
|---|---|---|---|
| Vaccine and Antibody | Volume (mm³) | (mm³) | still alive |
| HPV control vaccine + | 308.20 | 103.5 | 10 out of 10 |
| CD28-binding bsAb (10 mg/kg) | | | |
| HPV control vaccine + | 342.10 | 118.2 | 10 out of 10 |
| CD38 × CD28 bsAb (10 mg/kg) | | | |
| Melanocyte vaccine + | 354.55 | 214.20 | 10 out of 10 |
| CD28-binding bsAb (10 mg/kg) | | | |
| Melanocyte vaccine + | 0.00 | 0.00 | 11 out of 11 |
| CD38 × CD28 bsAb (10 mg/kg) | | | |

TABLE 14
Tumor Growth and Survival at Day 25

| | Mean Tumor | Tumor Volume SEM | Number mice |
|---|---|---|---|
| Vaccine and Antibody | Volume (mm³) | (mm³) | still alive |
| HPV control vaccine + | 780.80 | 231.00 | 10 out of 10 |
| CD28-binding bsAb (10 mg/kg) | | | |
| HPV control vaccine + | 844.50 | 345.70 | 10 out of 10 |
| CD38 × CD28 bsAb (10 mg/kg) | | | |
| Melanocyte vaccine + | N/A | N/A | 9 out of 10 |
| CD28-binding bsAb (10 mg/kg) | | | |
| Melanocyte vaccine + | 0.00 | 0.00 | 11 out of 11 |
| CD38 × CD28 bsAb (10 mg/kg) | | | |

TABLE 15
Tumor Growth and Survival at Day 30

| | Mean Tumor | Tumor Volume SEM | Number mice |
|---|---|---|---|
| Vaccine and Antibody | Volume (mm³) | (mm³) | still alive |
| HPV control vaccine + | N/A | N/A | 8 out of 10 |
| CD28-binding bsAb (10 mg/kg) | | | |
| HPV control vaccine + | N/A | N/A | 9 out of 10 |
| CD38 × CD28 bsAb (10 mg/kg) | | | |
| Melanocyte vaccine + | N/A | N/A | 9 out of 10 |
| CD28-binding bsAb (10 mg/kg) | | | |
| Melanocyte vaccine + | 0.00 | 0.00 | 11 out of 11 |
| CD38 × CD28 bsAb (10 mg/kg) | | | |

While monotherapy with a melanocyte mRNA vaccine (melanocyte mRNA vaccine plus 0028-binding control bispecific Ab) showed modest anti-tumor efficacy against B16F10.9/huBCMA/huCD38 tumors, combination treatment with melanocyte mRNA vaccine plus huCD38xhuCD28 bispecific antibody resulted in a more potent, combinatorial anti-tumor efficacy. The studies demonstrate that co-stimulation by a TAAxCD38 bispecific antibody enhances immunity against tumors induced by a cancer vaccine. Combination treatment with melanocyte vaccine and a huCD38xhuCD28 bsAb results in more potent, combinatorial anti-tumor efficacy that is superior to melanocyte vaccine treatment alone.

Table 16 sets forth the amino acid sequences corresponding to the mRNA vaccines used in this study and a study described in Example 2.

TABLE 16
Amino Acid Sequences Corresponding to mRNA vaccines

| Seq | | |
|---|---|---|
| ID No. | Sequence | Description |
| 1 | VTLCNGTYEGLLRRNKVGRNNEKLPTLSGGGGGGGGYVITTQHW | Melanocyte |
| | LSGGGGSGGGGFSHQGPAFVTWHRYHLLSGGGGSGGGGSVYDF | TRP-2 |
| | FVWLHYYSVGGGGSGGGGSLAPIGHNRMYNMVPFFPPVGGGGS | vaccine |
| | GGGGSPAILRRNIHSLSGGGGSGGGGLAFLQYRRLRKGYAPLMET | |
| | GLSGGGGSGGGGSTFSFRNALEGFDKADGTLDSQVMNLHNLAHS | |
| | FLNGTNALPHSAANDPVFVGGGGSGGGGSLGYNYAVDLSEEEAPV | |
| | WSTTL | |
| 2 | DLVLKRCLLHLAVIGALLAVGATKVPRNQDWLGVSRQLRTKAWNR | Melanocyte |
| | QLYPEWTEAQRLDCWRGGQVSLKVS | PMEL vaccine |
| 3 | FQDPQERPRKLPQLCTELQTTIHDIILECVYCKQQLLRREVYDFAFR | Control HPV |
| | DLCIVYRDGNPYAVCDKCLKFYSKISEYRHYCYSLYGTTLEQQYNK | vaccine |
| | PLCDLLIRCINCQKPLCPEEKQRHLDKKQRFHNIRGRWTGRCMSCC | |
| | RSSRTRRETQLGGGSSGGGSGHGDTPTLHEYMLDLQPETTDLYC | |
| | YEQLNDSSEEEDEIDGPAGQAEPDRAHYNIVTFCCKCDSTLRLCVQ | |
| | STHVDIRTLEDLLMGTLGIVCPICSQKP | |
| 4 | TQHWLGLLGPNGTQPQIANCSVYDFFVWLHYYSVRDTLLGPGRPY | Melanocyte |
| | KAIDFSHQGPAFVTWHRYHLLWLERSGGGGSGGGGSTFSFRNAL | TRP-2 + |
| | EGFDKADGTLDSQVMNLHNLAHSFLNGTNALPHSAANDPVFVSGG | PMEL vaccine |
| | GGSGGGGDLVLKRCLLHLAVIGALLAVGATKVPRNQDWLGVSRQL | |
| | RTKAWNRQLYPEWTEAQRLDCWRGGQVSLKVS | |

### Example 2: Robust Anti-Tumor Efficacy of a Combination of a Cancer mRNA Vaccine Delivered Prophylactically or Therapeutically and a TAAxCD28 Bispecific Antibody

This example relates to two in vivo studies demonstrating that combination therapy of a melanocyte antigen mRNA vaccine, delivered prophylactically or therapeutically, and a huPSMAxhuCD28 bispecific antibody drives a robust in vivo anti-tumor response in a syngeneic mouse model of melanoma. While monotherapy of a melanocyte mRNA vaccine delivered prophylactically showed modest anti-tumor efficacy, combination therapy of melanocyte mRNA vaccine, delivered prophylactically or therapeutically, and huPSMAxhuCD28 bispecific antibody resulted in potent, combinatorial anti-tumor efficacy.

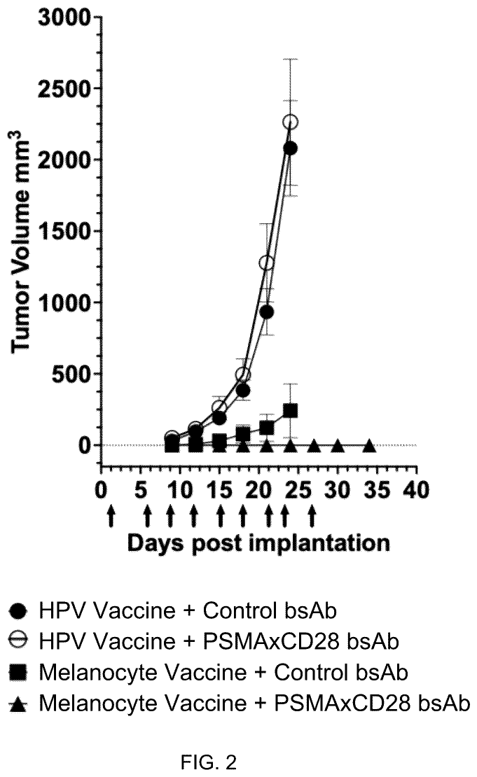

For both studies, C57BL/6 mice were engineered to express human CD28 (huCD28) and huPSMA (in addition to huCD3 and hu41BB) via Velocigene technology, as previously described (Valenzuela 2003). B16F10.9/huPSMA tumor cells endogenously express the murine (mu) melanocyte antigens muTRP-2 and muPMEL, and were engineered to express huPSMA cell-surface protein, which represents a tumor-specific antigen that is recognized by the huPSMAxhuCD28 antibody. The melanocyte mRNA vaccine used in these studies encodes immunogenic epitopes from muTRP-2 and huPMEL that stimulate T cell responses against muTRP-2 and muPMEL, which are presented by B16F10.9/huPSMA tumor cells.
Study 1: In this study, the anti-cancer vaccine was delivered prophylactically, i.e., before tumor implantation. Groups of 7 8-10-week-old huPSMA/huCD3/huCD28/hu41BB mice were vaccinated with the melanocyte mRNA vaccine 3 times, with each dose separated by 7 days. The mRNA vaccine was complexed with lipid nanoparticles (LNP) and delivered in two parts that were administered on two consecutive days. On the first day, mRNA encoding immunogenic epitopes from muTRP-2 (9 minigenes from muTRP-2) was administered i.v. (retro-orbitally). On the second day, mRNA encoding immunogenic epitopes from huPMEL (heteroclitic vaccine, 1 minigene from huPMEL) was administered i.v. (retro-orbitally). Thus, 5 μg of the muTRP-2 vaccine was administered on Day (−21), Day (−14), and Day (−7), and 5 μg of the huPMEL vaccine was administered on Day (−20), Day (−13) and Day (−6). As a negative control, an mRNA vaccine expressing immunogenic epitopes from the Human Papilloma Virus (HPV) oncogenes E6 and E7 was administered at the same schedule. On Day 0, mice were injected subcutaneously with 5×10⁵ B16F10.9/huPSMA tumor cells. On Day 1, the mice were administered either Control bsAb (one arm binding huCD28 and the other arm binding an unrelated antigen) or a huPSMAxhuCD28 bsAb (see U.S. Ser. No. 11/548,947) at 10 mg/kg intraperitoneally (i.p.). The mice received eight more doses of these bispecific antibodies on Days 6, 9, 12, 15, 18, 21, 24 and 27 for a total of nine doses. Tumor growth was assessed twice weekly over 35 days by caliper measurement, and tumor volume was calculated using the following formula: (length×width²)/2.
Study 1 Results: In the prophylactic setting, when the melanocyte vaccine was delivered before tumor cell implantation, combination therapy of melanocyte vaccine and PSMAxCD28 bsAb was remarkably effective at inhibiting melanoma tumor growth (FIG. 2; Tables 17-23). While mice administered a combination of HPV vaccine control and Control bsAb showed uninhibited tumor growth, mice administered the combination of melanocyte vaccine and PSMAxCD28 bsAb exhibited complete tumor rejection, with 100% of the mice becoming tumor-free by Day 28 (FIG. 2; Tables 17-23). Mice administered HPV vaccine control and PSMAxCD28 bsAb exhibited little inhibition of tumor growth, and mice administered of melanocyte vaccine and Control bsAb showed modest anti-tumor efficacy (FIG. 2; Tables 17-23). In this prophylactic setting, administration of the tumor-specific vaccine and PSMAxCD28 bsAb showed a strong synergistic effect, with the combination therapy of melanocyte vaccine and PSMAxCD28 bsAb exhibiting a much greater ability to inhibit tumor growth and promote complete tumor rejection than either agent administered as monotherapy.

Tables 17-23 set forth tumor growth and survival at Days 12, 15, 18, 21, 24, 27, and 30, respectively.

TABLE 17
Tumor Growth and Survival at Day 12

| | Mean Tumor | Tumor Volume SEM | Number mice |
|---|---|---|---|
| Vaccine and Antibody | Volume (mm³) | (mm³) | still alive |
| HPV control vaccine + | 95.22 | 34.81 | 7 out of 7 |
| CD28-binding bsAb (10 mg/kg) | | | |
| HPV control vaccine + | 114.55 | 32.09 | 7 out of 7 |
| PSMA × CD28 bsAb (10 mg/kg) | | | |
| Melanocyte vaccine + | 6.55 | 5.26 | 7 out of 7 |
| CD28-binding bsAb (10 mg/kg) | | | |
| Melanocyte vaccine + | 0.71 | 0.71 | 7 out of 7 |
| PSMA × CD28 bsAb (10 mg/kg) | | | |

TABLE 18
Tumor Growth and Survival at Day 15

| | Mean Tumor | Tumor Volume SEM | Number mice |
|---|---|---|---|
| Vaccine and Antibody | Volume (mm³) | (mm³) | still alive |
| HPV control vaccine + | 190.67 | 36.91 | 7 out of 7 |
| CD28-binding bsAb (10 mg/kg) | | | |
| HPV control vaccine + | 261.43 | 79.95 | 7 out of 7 |
| PSMA × CD28 bsAb (10 mg/kg) | | | |
| Melanocyte vaccine + | 31.21 | 27.52 | 7 out of 7 |
| CD28-binding bsAb (10 mg/kg) | | | |
| Melanocyte vaccine + | 0 | 0 | 7 out of 7 |
| PSMA × CD28 bsAb (10 mg/kg) | | | |

TABLE 19
Tumor Growth and Survival at Day 18

| | Mean Tumor | Tumor Volume SEM | Number mice |
|---|---|---|---|
| Vaccine and Antibody | Volume (mm³) | (mm³) | still alive |
| HPV control vaccine + | 384.45 | 70.41 | 7 out of 7 |
| CD28-binding bsAb (10 mg/kg) | | | |
| HPV control vaccine + | 493.93 | 112.02 | 7 out of 7 |
| PSMA × CD28 bsAb (10 mg/kg) | | | |
| Melanocyte vaccine + | 78.11 | 63.22 | 7 out of 7 |
| CD28-binding bsAb (10 mg/kg) | | | |
| Melanocyte vaccine + | 0 | 0 | 7 out of 7 |
| PSMA × CD28 bsAb (10 mg/kg) | | | |

TABLE 20
Tumor Growth and Survival at Day 21

| | Mean Tumor | Tumor Volume | Number mice |
|---|---|---|---|
| Vaccine and Antibody | Volume (mm³) | SEM (mm³) | still alive |
| HPV control vaccine + | 933.62 | 161.28 | 7 out of 7 |
| CD28-binding bsAb (10 mg/kg) | | | |
| HPV control vaccine + | 1277.02 | 274.98 | 7 out of 7 |
| PSMA × CD28 bsAb (10 mg/kg) | | | |
| Melanocyte vaccine + | 122.54 | 95.96 | 7 out of 7 |
| CD28-binding bsAb (10 mg/kg) | | | |
| Melanocyte vaccine + | 0 | 0 | 7 out of 7 |
| PSMA × CD28 bsAb (10 mg/kg) | | | |

TABLE 21
Tumor Growth and Survival at Day 24

| | Mean Tumor | Tumor Volume SEM | Number mice |
|---|---|---|---|
| Vaccine and Antibody | Volume (mm³) | (mm³) | still alive |
| HPV control vaccine + | 2080.65 | 334.45 | 6 out of 7 |
| CD28-binding bsAb (10 mg/kg) | | | |
| HPV control vaccine + | 2264.29 | 441.28 | 7 out of 7 |
| PSMA × CD28 bsAb (10 mg/kg) | | | |
| Melanocyte vaccine + | 242.89 | 189.16 | 7 out of 7 |
| CD28-binding bsAb (10 mg/kg) | | | |
| Melanocyte vaccine + | 0 | 0 | 7 out of 7 |
| PSMA × CD28 bsAb (10 mg/kg) | | | |

TABLE 22
Tumor Growth and Survival at Day 27

| | Mean Tumor | Tumor Volume SEM | Number mice |
|---|---|---|---|
| Vaccine and Antibody | Volume (mm³) | (mm³) | still alive |
| HPV control vaccine + | 2755.58 | 930.82 | 2 out of 7 |
| CD28-binding bsAb (10 mg/kg) | | | |
| HPV control vaccine + | 2477.48 | 148.98 | 3 out of 7 |
| PSMA × CD28 bsAb (10 mg/kg) | | | |
| Melanocyte vaccine + | 76.84 | 32.73 | 6 out of 7 |
| CD28-binding bsAb (10 mg/kg) | | | |
| Melanocyte vaccine + | 0 | 0 | 7 out 7 |
| PSMA × CD28 bsAb (10 mg/kg) | | | |

TABLE 23
Tumor Growth and Survival at Day 30

| | Mean Tumor | Tumor Volume SEM | Number mice |
|---|---|---|---|
| Vaccine and Antibody | Volume (mm³) | (mm³) | still alive |
| HPV control vaccine + | N/A | N/A | 0 out of 7 |
| CD28-binding bsAb (10 mg/kg) | | | |
| HPV control vaccine + | N/A | N/A | 0 out of 7 |
| PSMA × CD28 bsAb (10 mg/kg) | | | |
| Melanocyte vaccine + | 178.36 | 80.56 | 6 out of 7 |
| CD28-binding bsAb (10 mg/kg) | | | |
| Melanocyte vaccine + | 0 | 0 | 7 out of 7 |
| PSMA × CD28 bsAb (10 mg/kg) | | | |

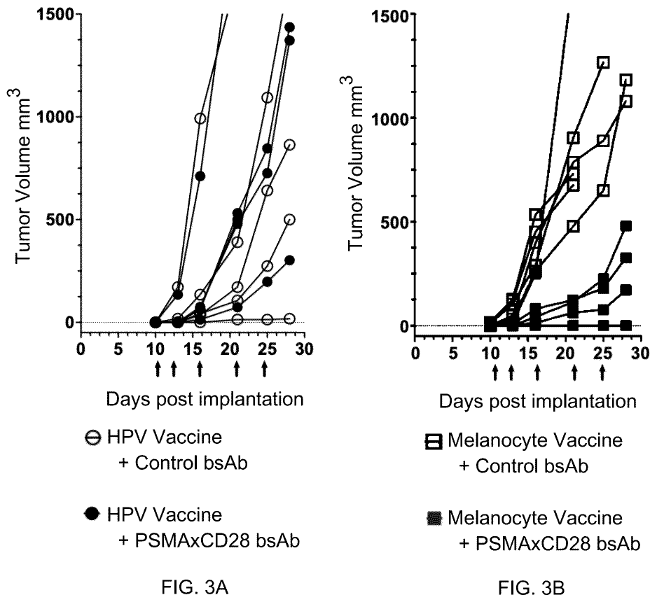

Study 2: In this study, the anti-cancer vaccine was delivered in a therapeutic setting, i.e., after tumor implantation. On Day 0, groups of 5 8-10-week-old huPSMA/huCD3/huCD28/hu41BB mice were injected subcutaneously with 5×10⁵ B16F10.9/huPSMA tumor cells. Mice were vaccinated starting on Day 1 with the melanocyte mRNA vaccine 3 times, with each dose separated by 7 days. In this study a muTRP-2+huPMEL melanocyte vaccine was used in which immunogenic sequences from muTRP2 (2 minigenes) and huPMEL (1 minigene) are combined into one single mRNA construct. Thus, 0.2 ug of the muTRP-2+huPMEL vaccine was administered on Days 1, 7, and 14. The mRNA vaccine was complexed with LNP and administered i.v. (retro-orbitally). As a negative control, a mRNA vaccine expressing immunogenic epitopes from the Human Papilloma Virus (HPV) oncogenes E6 and E7 was administered at the same schedule. On Day 10, the mice were administered either Control bsAb (one arm binding huCD28 and the other arm binding an unrelated antigen) or the huPSMAxhuCD28 bsAb at 10 mg/kg intraperitoneally (i.p.). The mice received four more doses of the bispecific antibodies on Days 13, 16, 21, and 25, for a total of five doses. Tumor growth was assessed twice weekly over 30 days by caliper measurement, and tumor volume was calculated using the following formula: (length×width²)/2.
Study 2 Results: In the therapeutic setting, when the melanocyte vaccine was delivered after tumor cell implantation, combination therapy of melanocyte vaccine and PSMAxCD28 bsAb exhibited marked inhibition of melanoma tumor growth (FIGS. 3A and 3B; Tables 24-30). In contrast, little tumor growth control was observed in response to either monotherapy, i.e., either HPV vaccine control and PSMAxCD28 bsAb or melanocyte vaccine and Control bsAb (FIGS. 3A and 3B; Tables 24-30). Thus, administration of the tumor-specific, melanocyte vaccine and a PSMAxCD28 bsAb showed a strong synergistic effect.

Tables 24-30 set forth tumor growth and survival at Days 10, 13, 16, 21, 25, 28, and 31, respectively.

TABLE 24
Tumor Growth and Survival at Day 10

| | Mean Tumor | Tumor Volume SEM | Number mice |
|---|---|---|---|
| Vaccine and Antibody | Volume (mm³) | (mm³) | still alive |
| HPV control vaccine + | 17.80 | 8.02 | 5 out of 5 |
| CD28-binding bsAb (10 mg/kg) | | | |
| HPV control vaccine + | 9.55 | 6.39 | 5 out of 5 |
| PSMA × CD28 bsAb (10 mg/kg) | | | |
| Melanocyte vaccine + | 7.01 | 3.13 | 5 out of 5 |
| CD28-binding bsAb (10 mg/kg) | | | |
| Melanocyte vaccine + | 0 | 0 | 5 out of 5 |
| PSMA × CD28 bsAb (10 mg/kg) | | | |

TABLE 25
Tumor Growth and Survival at Day 13

| | Mean Tumor | Tumor Volume SEM | Number mice |
|---|---|---|---|
| Vaccine and Antibody | Volume (mm³) | (mm³) | still alive |
| HPV control vaccine + | 64.30 | 0.92 | 5 out of 5 |
| CD28-binding bsAb (10 mg/kg) | | | |
| HPV control vaccine + | 66.41 | 0.85 | 5 out of 5 |
| PSMA × CD28 bsAb (10 mg/kg) | | | |
| Melanocyte vaccine + | 87.21 | 19.26 | 5 out of 5 |
| CD28-binding bsAb (10 mg/kg) | | | |
| Melanocyte vaccine + | 4.26 | 2.76 | 5 out of 5 |
| PSMA × CD28 bsAb (10 mg/kg) | | | |

TABLE 26
Tumor Growth and Survival at Day 16

| | Mean Tumor | Tumor Volume SEM | Number mice |
|---|---|---|---|
| Vaccine and Antibody | Volume (mm³) | (mm³) | still alive |
| HPV control vaccine + | 209.93 | 1.00 | 5 out o 5 |
| CD28-binding bsAb (10 mg/kg) | | | |
| HPV control vaccine + | 212.66 | 1.56 | 5 out of 5 |
| PSMA × CD28 bsAb (10 mg/kg) | | | |
| Melanocyte vaccine + | 387.71 | 50.41 | 5 out of 5 |
| CD28-binding bsAb (10 mg/kg) | | | |
| Melanocyte vaccine + | 77.41 | 45.32 | 5 out of 5 |
| PSMA × CD28 bsAb (10 mg/kg) | | | |

TABLE 27
Tumor Growth and Survival at Day 21

| | Mean Tumor | Tumor Volume | Number mice |
|---|---|---|---|
| Vaccine and Antibody | Volume (mm³) | SEM (mm³) | still alive |
| HPV control vaccine + | 512.62 | 2.27 | 4 out of 5 |
| CD28-binding bsAb (10 mg/kg) | | | |
| HPV control vaccine + | 417.22 | 1.71 | 4 out of 5 |
| PSMA × CD28 bsAb (10 mg/kg) | | | |
| Melanocyte vaccine + | 714.96 | 70.16 | 5 out of 5 |
| CD28-binding bsAb (10 mg/kg) | | | |
| Melanocyte vaccine + | 397.58 | 323.23 | 5 out of 5 |
| PSMA × CD28 bsAb (10 mg/kg) | | | |

TABLE 28
Tumor Growth and Survival at Day 25

| | Mean Tumor | Tumor Volume SEM | Number mice |
|---|---|---|---|
| Vaccine and Antibody | Volume (mm³) | (mm³) | still alive |
| HPV control vaccine + | 983 | 2.49 | 4 out of 5 |
| CD28-binding bsAb (10 mg/kg) | | | |
| HPV control vaccine + | 753 | 2.40 | 4 out of 5 |
| PSMA × CD28 bsAb (10 mg/kg) | | | |
| Melanocyte vaccine + | 935.91 | 155.64 | 3 out of 5 |
| CD28-binding bsAb (10 mg/kg) | | | |
| Melanocyte vaccine + | 452.09 | 333.97 | 5 out of 5 |
| PSMA × CD28 bsAb (10 mg/kg) | | | |

TABLE 29
Tumor Growth and Survival at Day 28

| | Mean Tumor | Tumor Volume SEM | Number mice |
|---|---|---|---|
| Vaccine and Antibody | Volume (mm³) | (mm³) | still alive |
| HPV control vaccine + | 989.90 | 2.80 | 3 out of 5 |
| CD28-binding bsAb (10 mg/kg) | | | |
| HPV control vaccine + | 278.32 | 0.60 | 2 out of 5 |
| PSMA × CD28 bsAb (10 mg/kg) | | | |
| Melanocyte vaccine + | 1131.50 | 51.50 | 2 out of 5 |
| CD28-binding bsAb (10 mg/kg) | | | |
| Melanocyte vaccine + | 630.81 | 394.45 | 5 out of 5 |
| PSMA × CD28 bsAb (10 mg/kg) | | | |

TABLE 30
Tumor Growth and Survival at Day 31

| | Mean Tumor | Tumor Volume SEM | Number mice |
|---|---|---|---|
| Vaccine and Antibody | Volume (mm³) | (mm³) | still alive |
| HPV control vaccine + | 1738.39 | 2.56 | 3 out of 5 |
| CD28-binding bsAb (10 mg/kg) | | | |
| HPV control vaccine + | 690.75 | 0.55 | 2 out of 5 |
| PSMA × CD28 bsAb (10 mg/kg) | | | |
| Melanocyte vaccine + | 1704.25 | 20.55 | 2 out of 5 |
| CD28-binding bsAb (10 mg/kg) | | | |
| Melanocyte vaccine + | 537.94 | 204.06 | 4 out of 5 |
| PSMA × CD28 bsAb (10 mg/kg) | | | |

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

### REFERENCES

Valenzuela et al., Nat Biotechnol. 2003; 21:652-659.

We claim:

1. A method for treating or inhibiting the growth of a tumor, comprising:
(a) selecting a subject with cancer; and
(b) administering to the subject a therapeutically effective amount of a cancer vaccine in combination with a therapeutically effective amount of a bispecific antigen-binding molecule, wherein the bispecific antigen-binding molecule comprises a first antigen-binding domain that binds specifically to a tumor-associated antigen (TAA) and a second antigen-binding domain that binds specifically to CD28,
wherein administration of the combination leads to increased anti-tumor efficacy, as compared to a subject administered the cancer vaccine as monotherapy.

2. A method for increasing the efficacy of a cancer vaccine, comprising:
(a) selecting a subject with cancer; and
(b) administering to the subject a therapeutically effective amount of a cancer vaccine in combination with a therapeutically effective amount of a bispecific antigen-binding molecule, wherein the bispecific antigen-binding molecule comprises a first antigen-binding domain that binds specifically to a tumor-associated antigen (TAA) and a second antigen-binding domain that binds specifically to CD28,
wherein administration of the combination leads to increased anti-tumor efficacy, as compared to a subject administered the cancer vaccine as monotherapy.

3. The method of claim 1 or 2, wherein the cancer vaccine comprises mRNA.

4. The method of any one of claims 1-3, wherein the TAA is selected from AFP, ALK, BAGE proteins, BCMA, BIRC5 (survivin), BIRC7, β-catenin, brc-abl, BRCA1, BORIS, CA9, carbonic anhydrase IX, caspase-8, CALR, CCR5, CD19, CD20 (MS4A1), CD22, CD30, CD38, CD40, CDK4, CEA, CTLA4, cyclin-B1, CYP1B1l, EGFR, EGFRvIII, ErbB2/Her2, ErbB3, ErbB4, ETV6-AML, EpCAM, EphA2, Fra-1, FOLR1, GAGE proteins, GD2, GD3, GloboH, glypican-3, GM3, gp100, Her2, HLA/B-raf, HLA/k-ras, HLA/MAGE-A3, hTERT, LMP2, MAGE proteins, MART-1, mesothelin, ML-IAP, Muc1, Muc2, Muc3, Muc4, Muc5, Muc16 (CA-125), MUM1, NA17, NY-BR1, NY-BR62, NY-BR85, NY-ESO1, OX40, p15, p53, PAP, PAX3, PAX5, PCTA-1, PLAC1, PRLR, PRAME, PSCA, PSMA (FOLH1), RAGE proteins, Ras, RGS5, Rho, SART-1, SART-3, STEAP1, STEAP2, TAG-72, TGF-β, TMPRSS2, Thompson-nouvelle antigen (Tn), TRP-1, TRP-2, tyrosinase, and uroplakin-3.

5. The method of any one of claims 1-4, wherein the cancer is selected from anal cancer, angiosarcoma, basal cell carcinoma, bladder cancer, bone cancer, brain cancer, breast cancer, a B-cell cancer, cervical cancer, cholangiocarcinoma, chondrosarcoma, colon cancer, colorectal cancer, cutaneous squamous cell carcinoma, endometrial cancer, esophageal cancer, glioblastoma multiforme, head and neck squamous cell cancer, hepatocellular carcinoma, kidney cancer, leukemia, liver cancer, lung cancer, lymphoma, melanoma, Merkel cell carcinoma, myeloma, non-small cell lung cancer, ovarian cancer, pancreatic cancer, prostate cancer, rectal cancer, salivary gland cancer, skin cancer, soft tissue sarcoma, stomach cancer, testicular cancer, and uterine cancer.

6. The method of any one of claims 1-5, wherein the cancer vaccine is autogene cevumeran.

7. The method of claim 6, wherein the cancer is pancreatic cancer.

8. The method of any one of claims 1-5, wherein the cancer vaccine is nadoferagene firadenovec.

9. The method of claim 8, wherein the cancer is bladder cancer.

10. The method of any one of claims 1-5, wherein the cancer vaccine is a personalized cancer mRNA vaccine.

11. The method of claims 1-5 or 10, wherein the cancer mRNA vaccine incorporates one or more subject-specific neoantigens.

12. The method of any one of claims 1-5, wherein the cancer vaccine is selected from BNT113, BNT111, BNT116, BNT122, BNT142, BNT152, BNT153, WGC-043, mRNA-5671/V941, mRNA-4157, mRNA-252, and CV9202.

13. The method of any one of claims 1-12, wherein administration of the combination produces a therapeutic effect selected from one or more of delay in tumor growth, reduction in tumor cell number, increase in tumor regression, prevention of tumor recurrence, prevention or inhibition of metastasis, and inhibition of metastatic tumor growth.

14. The method of any one of claims 1-12, wherein administration of the combination produces a therapeutic effect selected from one or more of increase in duration of survival, increase in likelihood of partial response, increase in likelihood of complete response, and elimination of the need for surgery.

15. The method of any one of claims 1-14, wherein the cancer vaccine is administered to the subject before or concurrently with the administration of the bispecific antigen-binding molecule.

16. The method of any one of claims 1-14, wherein the cancer vaccine is administered to the subject after the administration of the bispecific antigen-binding molecule.

17. The method of any one of claims 1-16, wherein the cancer vaccine is administered intravascularly, subcutaneously, intraperitoneally, or intratumorally.

18. The method of any one of claims 1-17, wherein the bispecific antigen-binding molecule is a bispecific antibody.

19. The method of any one of claims 1-18, further comprising administering an additional therapeutic agent or therapy to the subject.

20. The method of claim 19, wherein the additional therapeutic agent or therapy is selected from radiation, surgery, a 3-dioxygenase (IDO) inhibitor, an angiopoietin-2 (Ang2) inhibitor, an anti-inflammatory drug, an antibody to a tumor-specific antigen, an antibody-drug conjugate, a B and T lymphocyte attenuator (BTLA) inhibitor, a B7-H3 inhibitor, a B7-H4 inhibitor, Bacillus Calmette-Guerin vaccine, a CD3 inhibitor, a CD47 inhibitor, a chemotherapeutic agent, a cytotoxin, an epidermal growth factor receptor (EGFR) inhibitor, a galectin 9 (GAL9) inhibitor, granulocyte-macrophage colony-stimulating factor (GM-CSF), an IL-10 inhibitor, IL-2, IL-7, IL-12, IL-15, IL-21, indoleamine-2, an interleukin 4 receptor (IL-4R) inhibitor, an interleukin 6 receptor (IL-6R) inhibitor, a Killer-Cell Immunoglobulin-Like Receptor (KIR) inhibitor, a lymphocyte activation gene 3 (LAG3) inhibitor, a T cell immunoglobulin and mucin-domain containing-3 (TIM3) inhibitor, a PD-1 inhibitor, a T cell immunoreceptor with Ig and ITIM domains (TIGIT) inhibitor, a transforming growth factor beta (TGFβ) inhibitor, a V-domain immunoglobulin (Ig)-containing suppressor of T cell activation (VISTA) inhibitor, a vascular endothelial growth factor (VEGF) antagonist, and combinations thereof.
